(19) United States
(12) Patent Application Publication
(10) Pub. No.: US 2004/0077077 A1
(43) Pub. Date: Apr. 22, 2004

(54) Novel methods for the production of cloned mammals, mammals cloned according to the methods, and methods of use of same

(76) Inventors: Xiangzhong Yang, Storrs, CT (US); Jangwon Lee, Storrs, CT (US)

Correspondence Address: EDWARDS & ANGELL, LLP, P.O. BOX 55874, BOSTON, MA, 02205, US

(21) Appl. No.: 10/274,432

(22) Filed: Oct. 18, 2002

(51) Int. Cl. A01K 67/027; C12N 5/06

(52) U.S. Cl. 435/325; 435/354; 800/21

(57) ABSTRACT

As disclosed herein, the present invention is directed to new methods for the production of cloned mammals based on whole cell injection of the donor cells into an enucleated oocyte to form a reconstructed oocyte. The present invention relates to improved methods for the cloning of transgenic mammals, the cloned mammals, and methods for use of the cloned transgenic mammals.

### FIELD OF THE INVENTION

The present invention relates to novel methods for production of cloned mammals based upon whole cell intracytoplasmic microinjection. More specifically, the present invention relates to novel methods for the cloning of transgenic mammals, the cloned mammals, and methods for use of the cloned transgenic mammals.

### BACKGROUND OF THE INVENTION

Since the birth of a cloned sheep from an adult somatic cell in 1997, there have been two primary procedures developed to introduce donor cell nuclei into enucleated oocytes in order to produce cloned mammals by the method of nuclear transfer (NT). The cell fusion method, which involves placing a donor cell in the perivitelline space of an enucleated recipient oocyte and fusing the donor and the recipient cell with electrical pulses, has been used to generate cloned sheep [Wilmut, I., et al., “Viable offspring derived form fetal and adult mammalian cells,” Nature 385: 810-813 (1997)], cattle [Cibelli, J. B. et al Cloned transgenic calves produced from nonquiescent fetal fibroblasts,” Science 280: 1256-1258 (1998); Kubota, C. et al., “Six cloned calves produced from adult fibroblast cells after long-term culture,” Proc. Nat'l. Acad. Sci. USA 97: 990-995 (2000)], goats [Baguisi, A. et al., “Production of goats by somatic cell nuclear transfer,” Nat. Biotechnology 17: 456-461 (1999)], as well as pigs [Polejaeva, I. A., et al., “Cloned pigs produced by nuclear transfer from adult somatic cells,” Nature 407: 86-90 (2000); Betthauser, J. et al., “Production of cloned pigs from in vitro systems,” Nat. Biotechnology 18: 1055-1059 (2000)].

Subsequently, a distinctive non-fusion method, in which nuclear material from donor cells is isolated and injected into enucleated oocytes by piezo-actuated microinjection (nucleus injection method), was developed and cloned mice and pigs have been generated [Wakayama, T., et al., “Full-term development of mice from enucleated oocytes injected with cumulus cell nuclei,” Nature 394: 369-374 (1998); Onishi, A., et al., “Pig cloning by microinjection of fetal fibroblast nuclei,” Science 289: 1188-1190 (2000)]. Most higher animals cloned to date have been produced by the cell fusion method. In general, information gathered to date suggests a wide variety of different animal species can be cloned by nuclear transplantation. However, the efficiency of both of these NT procedures remains low and time-consuming and the percentage of live offspring does not exceed 3% regardless of the species. The low efficiency of these methods is mainly caused by the low fusion rate or by damage to the nuclear material during extraction and/or injection. Low percentage of offspring born from embryos produced by current NT techniques suggests an ongoing, compelling need for alternative, less damaging procedures for creation of cloned embryos.

A large number of nuclear transfer studies have made use of embryonic cells or ovary cells as donor cells. The embryonic stem cell has been found to be a particularly useful cell as a donor cell in that it supports better development of enucleated oocytes to term. Genetic manipulation of mouse embryonic stem cells has revolutionized mouse genetic research. Unfortunately, embryonic stem cells are not readily available in other species.

It was not until the mid-1990's that reports of nuclear transfer from cultured cell lines arose. These reports (See, e.g., Wilmut et al., Nature (London) 385, 810-813) (1997)) suggest the usefulness of donor cells derived not only from embryos, but also those derived from differentiated somatic cells from blastocysts, fetuses or adult animals. Somatic cells derived from non-embryonic related tissues (hereinafter referred to as, somatic cells”) were not found to be useful as donor cells in producing viable animal clones. In fact, as stated in U.S. Pat. No. 5,945,577 to Stice et al., until the late 1990s it was widely believed that only embryonic or undifferentiated cell types could direct any sort of fetal development in nuclear transfer techniques. U.S. Pat. No. 5,945,577 to Stice et al., teaches advanced embryonic and fetal development from nuclear transfers from differentiated donor somatic cells to enucleated oocytes. U.S. Pat. No. 6,011,197 to Strelchenko et al., states that fibroblasts from a fibroblast cell culture derived from an adult ear punch may be used as nuclear donors in a nuclear transfer process. Both references, however, fail to demonstrate any viable animals being produced by their methodologies with somatic cell nuclei donation.

Most cloning efforts have been focused on the production of transgenic animals by utilizing genotypes that are defined by a particular genetic modification. However, there is also a considerable demand for cloning animals with inherent genetic value, as developed in traditional breeding programs based on Mendelian genetic principles. For examples, prized bulls or champion thoroughbred horses can be cloned to provide a supply of the animal's valuable genetic material at a level not possible through creation of sperm banks. Also, the value of females of the species can be better exploited through use of somatic cells from females with desirable phenotypic traits in cloning. In other cases, household pets for example, the demand to replicate a specific genotype is also increasing. Animals are also targeted for cloning because they are extremely rare, e.g. endangered species. In some cases, the animals identified as candidates for cloning are sterile, infertile, or even deceased.

Ultimately, there will be many important therapeutic benefits to new and alternative methods of cloning technology, including the production of genetically modified pigs for the transplantation of organs from one species to another, defined as xenotransplantation. Another therapeutic benefit derived from efficient cloning technology is the potential to present a promising solution to the inadequate supply of human organs. Currently, cloning a calf costs approximately $20,000 (see, for example, information available at www.cyagra.com). There is currently no listed price for cloning of a pig and the estimated cost is expected to be similar if not higher than that for cattle because pig cloning to date is much more inefficient than cattle cloning. Yet, pigs are the preferred donors for xenotransplantation. Pigs' organs that are rendered immunologically compatible with humans through genetic engineering techniques are best produced through cloning by nuclear transfer (NT) using genetically modified cells. [Lai, L., et al., “Production of α-1,3-Galactosyltransferase Knockout Pigs by Nuclear Transfer Cloning,” Science 295: 1089-1092 (2002); Dai, Y., et al., “Targeted disruption of the alpha 1,3-galactosyltransferase gene in cloned pigs,” Nat. Biotechnology 20: 251-255 (2002)]. The efficiency of pig cloning using currently available techniques, however, remains very low, time-consuming and constitutes a major impediment to the development of genetically modified pigs as xenotransplantation donors.

Transgenic animals, such as mice, are advantageous for the study of particular diseases. For example, a particular gene can be turned on or knocked-out, resulting in an animal with a specific disease state. This transgenic animal and its clones may then provide models for drug design. See Krieger, et al, U.S. Pat. No. 6,437,215. There also is a need for the efficient production of transgenic mammals other than mice. See DeBoer, et al., U.S. Pat. No. 5,633,076. Particularly, there is a need for the production of animals that exhibit desirable phenotypical traits. One example is for sheep that were engineered to produce the human blood-clotting protein factor IX in their milk. In addition, there is the potential and the need to create bovine species capable of producing recombinant polypeptides in milk. In another example, increases in milk production on a wide scale can be achieved using genetically superior sires.

Moreover, transgenic, cloned animal tissue can be used to treat diseases. Thus, another aspect of therapeutic cloning is the ability to produce genetically matched cells to a person's immune system so that their immune system will not reject a tissue or organ as foreign. Stem cells derived from this method can theoretically be transformed into any kind of tissue and match the recipient's genetic profile.

Research suggests that cloning human embryos for medical research could yield promising therapies for a myriad of diseases. See, Pearson, Nature, Jun. 21, 2002. Perhaps the most far-reaching potential application of human stem cells is the generation of cells and tissues that could be used for cell/tissue therapies. Many diseases and disorders result from disruption of cellular function or destruction of tissues of the body. A typical solution is to use donated organs and tissues to replace the ailing or destroyed tissue. A problem to overcome is the fact that there is often a greater patient need for donated organs than there are organs available for transplantation. The present invention addresses this need. Stem cells, stimulated to develop into specialized cells, offer the possibility of a renewable source of replacement cells and tissues to treat a myriad of diseases, conditions, and disabilities including Parkinson's and Alzheimer's diseases, spinal cord injury, stroke, burns, heart disease, diabetes, osteoarthritis and rheumatoid arthritis.

In addition to creating xenotransplantation donors through cloning with genetically modified donor cells, the increased efficiency and reduced costs associated with the practice of the present invention should enable the creation of cloned animals as bioreactors to produce proteins of potential value expressed from genes introduced into the cloned animal through genetic engineering techniques. Thus, this invention is useful for transgenic as well as non-transgenically modified animals.

Defined Terms:

Activation: by the term “activation” it is meant to refer to any materials and methods useful for stimulating a cell to divide before, during, and after a nuclear transfer step;

Animal Clone: a viable animal having a genome that is substantially similar or identical to the genome of another animal and which is produced by other than fusion of a sperm and nucleated oocyte. By “substantially similar” it is meant that the genes differ by copy error differences that normally occur during the replication of DNA;

Clone: a biomass having a nuclear DNA sequence that is substantially similar to or identical, to the nuclear DNA sequence of another biomass (such as a cell, an organ, a fetus, or an animal etc.). By “substantially similar” it is meant that the two sequences may differ by copy error differences that normally occur during the replication of a nuclear DNA;

Cloning Efficiency: the efficiency of production of embryo or an animal clone from a cybrid;

Cumulus Cell: any cultured or non-cultured cell isolated from cells and/or tissue surrounding an oocyte;

Embryo: a developing cell mass that has not implanted into the uterus of maternal host; by the term “embryo” it is meant to include a fertilized oocyte, a cybrid, a pre-implantation stage developing cell mass, etc.;

Fetus: a developing cell mass that has implanted into the uterus of material host;

Fibroblast: a cell-type present in vertebrate connective tissue that secretes tropocollagen and mucopolysaccharides which constitute the connective tissue ground substance. Fibroblast cells normally stain positive for vimentin and negative for cytokeratin stains;

Fibroblast-like Cell: cultured cells that have a distinct flattened morphology and are capable of growing within monolayers in culture;

Fusion: the combination of portions of lipid membranes corresponding to the cell nuclear donor and the recipient oocyte;

Genetically-Altered Animal: an animal carrying a gene mutation introduced by genetic engineering techniques;

Genetically-Altered Cell: by “genetically-altered cell” it is meant a cell carrying a gene mutation introduced by genetic engineering techniques;

Modified Nuclear DNA: nuclear deoxyribonucleic acid that has been manipulated by one or more recombinant DNA techniques;

Somatic Cell: a somatic cell that is derived from a source other reproductive cells such as the sperm or oocytes (see bellow).

Nuclear Transfer: Introducing a full complement of nuclear DNA from one cell into an enucleated cell.;

Pluripotent: the capacity of a cell to differentiate into a sub-population of cells within a developing cell mass but not to give rise to all of the cells in such cell mass, such as an embryo, fetus or animal;

Quiescent Cell: a cell that is not dividing;

Reprogramming: the materials and methods that can convert a non-totipotent cell into a totipotent cell;

Serum Starve: culturing cells in a medium comprising a serum concentration sufficiently low to render cultured cells quiescent;

Somatic Cell: a cell other than a germ cell;

Term Animal: an animal capable of surviving one or more weeks outside of the environment where it developed (e.g., uterus) without the need for life support or medical intervention; by “full term animal” it is meant a term animal which is physiologically developed within the norms for neonates of such animals and delivered at the normal due date;

Totipotent: the capacity of a cell to give rise to all of the cells in a developing cell mass, such as an embryo, fetus or animal;

Transgenic Animal: an animal with a genome produced in whole or in part by artificial genetic manipulation means;

Ungulate: a four-legged animal having hooves;

Viable Animal: an animal capable of surviving for more than 365 days outside of a host animal without the need for artificial life support or medical intervention.

### SUMMARY OF THE INVENTION

An object of this invention is to provide a new method for the production of a reconstructed oocyte. The new method includes the selection of one or more oocytes from a mammal of a specific species and enucleating the oocytes. Then, one or more somatic donor cells are selected from a donor cell source and a whole cell from the donor cells is injected into an enucleated oocyte to form a reconstructed oocyte. The preferred method involves culturing the reconstructed oocyte under conditions sufficient to insure development of the reconstructed oocyte to a further developmental stage.

In an embodiment of the invention, the donor cells are either cumulus cells, mural granulosa cells, or fibroblast cells. Preferably, the donor cell source is a stable cell line. The donor cell source can be an embryo or fetal tissue. More preferably, the donor cell source is a mammal that has reached a developmental stage of independent viability. The species of the mammal can be pig, rabbit, cattle, goat or mouse.

In another embodiment of the invention, the mammal is a transgenic mammal.

Another embodiment includes the centrifugation of the donor cells prior to enucleation. Yet another embodiment includes the step of activating the reconstructed oocyte at a time subsequent to formation of the reconstructed oocyte sufficient to result in optimization of cloning efficiency. Preferably, the oocyte is activated by electrical stimulation. More preferably, the oocyte has minimum exposure to ultraviolet radiation. Even more preferably, the activation step occurs from 0 to 10 hours after injection of the donor cell into the enucleated oocyte. Still even more preferably, activation occurs from about 1 to 6 hours after injection.

In another embodiment, the method includes the additional step of conditioning the donor cells prior to activation. Preferably, the conditioning of donor cells is achieved by subjecting the oocyte to a prolonged period of time prior to activation of the reconstructed oocyte. Preferably, the period of time is 0 to 10 hours. Still more preferably, the conditioning is for a period of 1 to 6 hours.

In yet another embodiment, a cloned mammal is produced from a reconstructed oocyte using this method. In another embodiment, a stable cell line is derived from a reconstructed oocyte. An embryo, stem cell, tissue, organ, or combination thereof is developed from the reconstructed oocyte.

In another embodiment, the method involves the altering of one or more nucleotide sequences of the donor cell by genetic engineering techniques. Preferably, a cloned mammal is derived from this genetically altered donor cell. Preferably, a stable cell line, embryo, stem cell, tissue, or organ is developed from this genetically altered donor cell.

Still more preferably, the cloned mammal displays a desirable phenotypic trait conferred on it through the altered nucleotide sequence. Preferably, the one or more desirable phenotypic traits include a reduced immunostimulatory effect on a pre-selected potential xenotransplantation organ, tissue, or cell recipient. More preferably, the phenotypic trait is a pharmaceutically active species. Specifically, the pharmaceutically active species are therapeutic proteins.

The present invention also provides a method for the production of donor material cells, tissue, or organs for xenotransplantation. Preferably the method includes the steps of producing a cloned donor source, and harvesting the cells, tissue, or one or more organs from the cloned donor source. Moreover, the method further comprises altering at least one nucleotide sequence of one or more cells derived from the donor material by genetic engineering techniques.

In yet another embodiment, the present invention provides a method for the production of donor cells, tissues, or organs for xenotransplantation, comprising production of the cloned donor mammal by altering one or more nucleotide sequences of the donor cell by genetic altering techniques, and harvesting the cell, tissue, or organ from the cloned mammal for xenotransplantation.

In another embodiment, the present invention provides a method for the production of one or more potentially therapeutic proteins by producing a genetically altered cloned mammal by altering one or more nucleotide sequences of the donor cell, wherein the desirable phenotypic trait is the expression of one or more of the proteins, and extracting the proteins from the cloned mammal.

In a preferred embodiment, the donor cell is obtained from a mammal of an endangered species. The donor cell can also be from an animal that displays enhanced value as a livestock animal.

The donor cells are from a mammal of the same species of the recipient oocyte, or they can be from a mammal of a different species from the recipient oocyte. Preferably, the developmental stage to which the reconstructed oocyte is developed is an embryo stage. Moreover, the method involves transplantation of the embryo into a surrogate mother. More preferably, the surrogate mother is maintained under conditions sufficient to insure the development of the embryo into a fetus capable of sustaining life outside the surrogate mother, and delivering the developed fetus to produce a cloned animal.

The present invention is in principle applicable to all animals, including birds, amphibians and fish species. However, its greatest commercial usefulness presently envisioned is for non-human mammals. Its applicability extends not only to the family of ruminants belonging to the genus Bos (so called “bovines” which include cattle, oxen, sheep, and goats) but to other ungulates such as camels, pigs and water buffalo.

### BRIEF DESCRIPTION OF THE DRAWINGS

FIG. 1, in five panels A through E, depicts events following whole cell injection.

FIG. 2 is a comparative bar chart representing the blastocyst development of cloned embryos from cumulus, mural granulosa, and fibroblast cell at specific time intervals for activation after whole cell injection.

FIG. 3 is provided in two panels A and B; FIG. 3A is a photograph of three cloned pigs produced according to the methods of the present invention; FIG. 3B is a PCR assay for the αLA-pLF and αLA-hFIX double transgenes.

FIG. 4, in four panels (A through D), depicts the whole cell injection procedure and cloned embryos obtained therefrom.

### DETAILED DESCRIPTION OF THE INVENTION

The present invention provides a new approach to the production of cloned mammals. The present inventors have developed a method for the direct injection of an entire donor cell into the cytoplasm of an oocyte whose chromosomes have been removed. This technique provides significant advantages over the approaches currently available in the prior art. Among other advantages, the method of the present invention will: 1) save time and labor during the nuclear transfer process essential to successful cloning; 2) reduce the extent of oocyte manipulation required in the cloning process; and 3) improve the resulting efficiency of cloning. Through the practice of the method of the present invention, significant progress can be made toward large-scale production of cloned animals, including laboratory, domestic and livestock animals (including genetically modified species for xenotransplantation), as well as for preservation of endangered species. The practice of the present invention significantly reduces the time oocytes are manipulated during nuclear transfer and will improve the efficiency of the NT technology. Furthermore, only conventional micromanipulation equipment is needed to apply this invention to nuclear transfer as opposed to the nuclei injection technique which requires the use of a piezo-drill.

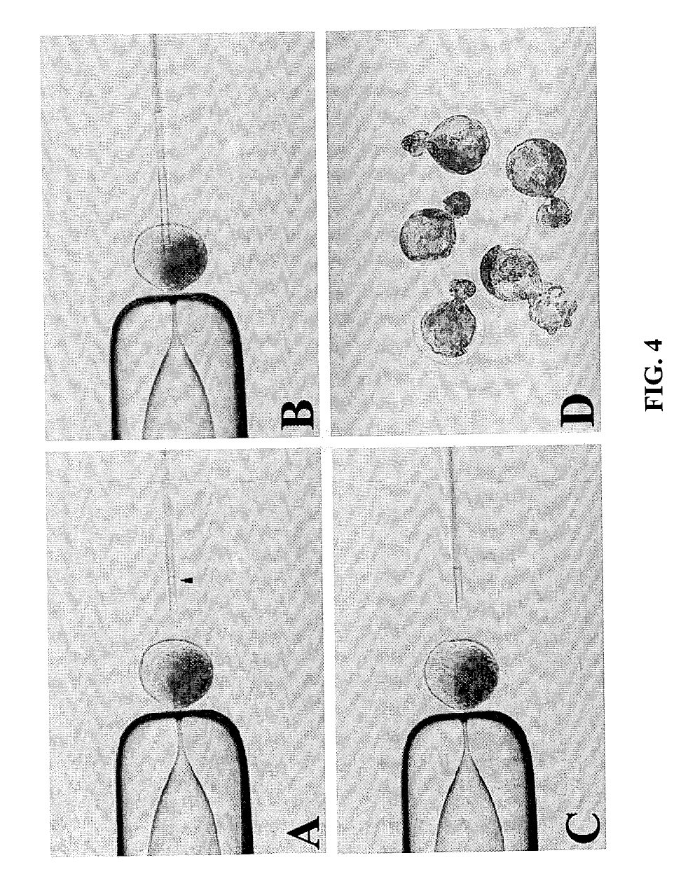

The improved efficiency of the method of the present invention is indicated by the higher development rates of cloned embryos (37% vs. approximately 10% in prior art reports) and by the high pregnancy rates, despite the fact that a relatively small number of embryos were transferred into each recipient (70-80 embryos vs. 100-300 embryos/recipient in previous reports). The higher efficiency of the whole cell injection technique can be attributed to the following: 1) whole cell injection assured delivery of DNA into each injected oocyte and thus avoided low fusion rates and potential damage to nuclear material during isolation and transfer; 2) the injection of a whole cell assured delivery of all cellular components to the enucleated oocytes; components of the donor cells may prove to be important for later development in pigs; the microtubule-organizing center (MTOC), for example, is needed during natural fertilization in most mammals except for the mouse; and 3) whole cell injection eliminates the fusion step which significantly reduces the manipulation time required for nuclear transfer and is therefore beneficial to embryo development. (See FIG. 4).

Oocytes are typically isolated from either oviducts and/or ovaries of live animals, although they may be retrieved from deceased animals as well. Oocytes are typically matured in a variety of medium known to those of ordinary skill in the art prior to enucleation. Generally the oocytes used in nuclear transfer techniques are in the metaphase II cell-cycle stage.

It is well known to those of ordinary skill in the art that enucleation of the oocyte can be performed by a number of techniques, including aspiration (Smith & Wilmut, Biol. Reprod., 40: 1027-1035 (1989)), by use of DNA-specific fluorochromes (See, e.g., Tusnoda et al., J. Reprod. Fertil. 82: 173 (1988)), and irradiation with ultraviolet light (See, e.g., Gurdon, Q. J. Microsc. Soc., 101: 299-311 (1960)). Enucleation may also be effected by other methods known in the art, such as described in U.S. Pat. No. 4,994,384, herein incorporated by reference. The oocyte can be exposed to a medium containing a microfilament disrupting agent or tubulin-disrupting agent prior to and during, enucleation. Disruption of the microfilaments imparts relative fluidity to the cell membrane and underlying cortical cytoplasm such that a portion of the oocyte enclosed within the membrane can be aspirated into a pipette. Successful enucleation may be confirmed by Hoechst 3342 fluorescent staining of the presumed cytoplasts or of the karyoplasts (elimination method, see 0035). Enucleation may also be performed by other techniques well known to those of ordinary skill in the art. For example, enucleation may involve the removal of the metaphase chromosomes from mature oocytes typically by aspirating the polar body and the adjacent cytoplasm. During the enucleation procedure oocytes may be exposed to 5 μg/mL Hoechst 33342 (plus 5 μg/mL cytochalasin B) for 5-10 minutes followed by enucleation manipulation under a fluorescent microscope.

An improvement over prior art, not directly related to whole cell injection, in the practice of the present invention includes the use of a non-invasive enucleation method in the absence of DNA staining and UV light exposure. Additionally, centrifugation of the oocytes prior to enucleation permitted more precise identification of the polar bodies. This technique also permits a high enucleation rate (78%, n=1300), the removal of minimal cytoplasm, and an improved ability to confirm that the injected donor cell is in the cytoplasm.

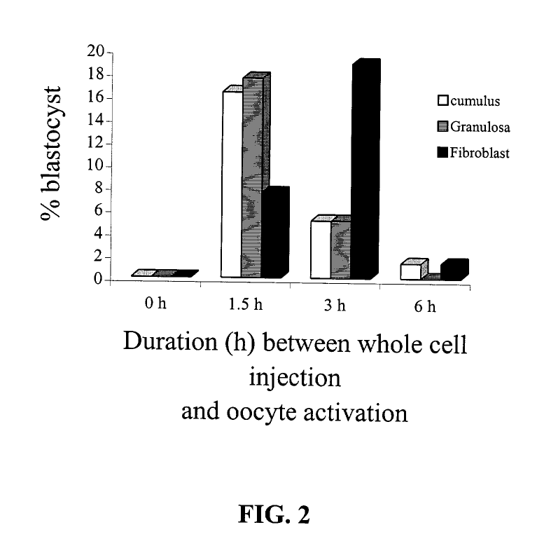

The method of the present invention for the injection of whole cells reduced the manipulation time of donor cells and recipient oocytes as compared to that of current NT procedures, through bypassing cell fusion and breakdown of the donor cell membrane. Different donor cell types vary in their “conditioning” requirement following whole cell injection. Also, different fusion rates are associated with fibroblast and cumulus cells in the cell fusion method of cloning (FIG. 2). Both of these phenomena may also be attributable to the differences in the membrane properties between these two cell types.

Centrifugation of oocytes and avoiding UV exposure during enucleation can further improve development. All of these improvements significantly reduced the overall damage to the cloned embryos, and greatly improved cloning efficiency. Prior to the present invention, a cybrid would typically be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated cybrids, or embryos, are typically cultured in medium well known to those of ordinary skill in the art, and include, without limitation, Tissue Culture Medium-199 (TCM-199)+10% fetal calf serum, Tyrodes-Albumin-Lactate-Pyruvate (TALP), Ham's F-10+10% fetal calf serum (FCS), synthetic oviductal fluid (“SOF”), B₂, CR₁ₐₐ medium and high potassium simplex medium (“KSOM”).

Cultured donor cells may be genetically altered by methods well-known to those of ordinary skill in the art. See, Molecular Cloning a Laboratory Manual, 2nd Ed., 1989, Sambrook, Fritsch and Maniatis, Cold Spring Harbor Laboratory Press; U.S. Pat. No. 5,612,205, Kay et al., issued Mar. 18, 1997; U.S. Pat. No. 5,633,067, to DeBoer et al., issued May 27, 1997. Any known method for inserting, deleting or modifying a desired gene from a mammalian cell may be used to alter the nuclear donor. Included is the technique of homologous recombination, which allows the insertion, deletion or modification of a gene or genes at specific site or sites in the cell genome. Examples for modifying a target DNA genome by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

The present invention can also selectively target gene changes in the genome of the donor cell, or selectively turn-off genes, using gene alteration and “knock-out” methods well known in the art. By gene targeting it is meant not only the inactivation of a gene but also altering of gene activity in any purposeful manner. Nuclei from such genetically-altered donor cells can then be used in nuclear transfer techniques as described herein to ultimately produce viable animals carrying the targeted genetic changes in their genomes. Animals produced using such gene targeting and cloning technique can be used to determine the function of a particular blocked gene, the importance of the conservation of a gene sequence, and as models for disease states, as well as for other purposes readily apparent to one of ordinary skill in the art. For example, the gene(s) responsible for certain immunological recognition proteins might be altered such that tissue from the host animal might be immunologically-acceptable by other animals (such as pig tissue being used in humans), or a gene(s) altered to produce a more commercially acceptable animal (e.g., a cow that produces more milk).

In one aspect of the invention there is provided a process by which genetically-altered and non-genetically altered animals may be produced, such process comprising the steps of: (a) isolating a diploid donor cell; (b) culturing the diploid donor cell for more than 10 doublings, preferably more than about 20 doublings, and yet more preferably more than 30 doublings, on a medium constituted such that the diploid donor cell multiplies; (c) optionally altering, preferably in a targeted manner, the genome of one or more cells of the diploid donor cells of step (b); (d) optionally screening and selecting from the cells of step (c) stable desired mutants; (e) reconstituting an embryo employing the nuclei transfer techniques using nuclei from the cells of step (b), or optionally steps (c) or (d); (f) culturing the embryo in vivo or in vitro to a blastocyst; (g) optionally screening and selecting from the blastocysts of step (f) stable desired mutants; (h) transfer of the blastocyst to medium capable of allowing the blastocyst to develop into a term animal. A particularly preferred donor cell is the fibroblast or fibroblast-like cell. Fibroblast cells may be collected from an ear (other part of the body) skin biopsy. In a method of preparation found advantageous, the tissue biopsy is cut into small pieces (3 mm²) and the pieces as tissue explants are cultured in DMEM (Gibco, 15) plus 10% fetal bovine serum (FBS) and antibiotics (Gibco, cat#15240-013) at 37.5° C. in a humidified atmosphere of 5% CO₂ and 95% air. After a week in culture, fibroblast cell monolayers form around the tissue explants. The explants are then removed to start new culture and the fibroblast cells are harvested weekly for freezing. For long term storage, the cultured cells may be collected following trypsin treatment, frozen in 10% dimethyl sulfoxide (Sigma) and stored in liquid nitrogen. Upon use for nuclear transfer, cells are thawed and cultured to confluency for passage. For each passage (estimated 2 cell doublings per passage), cells are cultured until confluent, disaggregated by incubation in a 0.1% (w/v) trypsin (Difco) and EDTA (Nacalai) solution for 1 min at 37° C. and allocated to three new dishes for further passaging. Normally, each passage lasts about six days.

The activated cybrids or embryos are preferably cultured on a suitable medium prior to implantation in the host, e.g., uterus. It is preferred that that the activated cybrid be cultured until greater than a 2-cell development stage. In one example, embryos are cultured in a CR1 aa medium for 48 hours at 38.5° C. in a humidified atmosphere at 5% CO₂, 5% O₂ and 90% N₂. Cleaved embryos may be cultured further in CR1aa medium supplemented with 5% FBS with cumulus-cell co-culture for 5 days. Blastocysts may be transferred non-surgically or surgically into the uterus of a synchronized recipient. Other medium may also be employed using techniques and media well-known to those of ordinary skill in the art. In one procedure, cloned embryos are washed three times with fresh KSOM and cultured in KSOM with 0.1% BSA for 4 days and subsequently with 1% BSA for an additional 3 days, under 5% CO₂, 5% O₂ and 90% N₂ at 39° C. Embryo development is examined and graded by standard procedures known in the art. Cleavage rates are recorded on day 2 and cleaved embryos are cultured further for 7 days. On day seven, blastocyst development is recorded and one or two embryos, pending availability of embryos and/or animals, is transferred non-surgically into the uterus of each synchronized foster mother.

Foster mothers preferably are examined for pregnancy by rectal palpation or ultrasonography periodically, such as on days 40, 60, 90 and 120 of gestation. Careful observations and continuous ultrasound monitoring (monthly) preferably is made throughout pregnancy to evaluate embryonic loss at various stages of gestation. Any aborted fetuses should be harvested, if possible, for DNA typing to confirm clone status as well as routine pathological examinations.

In order to more clearly describe the subject invention, the following examples are set forth along with the materials and methods used to undertake the same. The examples below are non-limiting and are merely representative of various aspects and features of the present invention.

### EXAMPLES

### Example 1

### Preparation of Donor Cells and Recipient Oocytes

In Vitro Maturation of Pig Oocytes.

Ovaries of prepubertal (or postpubertal) gilts were obtained from a local slaughterhouse. Oocytes were aspirated from antral follicles (3-7 mm in diameter) and cultured in a 100-μL droplet of maturation medium (BSA-free NCSU23 with 10% porcine follicular fluid, 0.1 mg/mL cysteine, 1% MEM non essential amino acid and 0.2 mM pyruvate) with hormonal supplementation (2 μg/mL Folltropin-V, Vetrepharm, Ontario, Canada) at 38.5° C. under 5% CO₂ in air for 44 hours.

Preparation of Adult Somatic Donor Cells.

Fresh cumulus cells were obtained by stripping in vitro matured oocytes in TL-HEPES supplemented with 0.1% hyaluronidase and washing three times in TL-HEPES with 0.4% BSA.

Mural granulosa cells were collected from antral follicles (3-7 mm in diameter) during oocyte aspiration. Isolated mural granulosa cells were washed with TL-HEPES and then approximately 1×10⁷ cells were plated in 60 mm culture dishes containing DMEM supplemented with 10% fetal calf serum and 1% antibiotic-antimycotic. Cultures were established by plating cells at a high density, after which they were allowed to reach confluency. The cells were routinely maintained on dishes until passage six and then were stored frozen as described below.

Fibroblast cell lines were established from skin samples taken from pig ear biopsies of a transgenic sow that expressed two transgenes—porcine lactoferrin (pLF) and human Factor IX (hFIX). Briefly, tissue pieces were rinsed in 95% ethanol and placed in phosphate buffered saline (PBS) supplemented with penicillin (100 IU/mL) and streptomycin (100 μg/mL) and minced into 1-2 mm pieces. Approximately 5 pieces were cultured in 2 mL of Dulbecco's Modified Eagle's Medium (DMEM) with 10% FBS and allowed to settle on the bottom of 65-mm tissue culture plates (Falcon) in a humidified 38.5° C. incubator with 5% CO₂. Cultures were fed every five days and tissue explants were removed and replated every ten days. The resulting monolayers were harvested by trypsin (0.05%) and EDTA (0.02%) treatment for 5 min.

For passaging, trypsinized cells were washed with PBS and replated at a 1:3 dilution. Cells were then passaged 10 times as confluence was reached, usually on day 4 or 5 after plating. Granulosa and fibroblast cells, at different passages, were collected after trypsin treatment, frozen in DMEM supplemented with 30% serum and 15% glycerol, and stored at −80° C. For whole cell injection, granulosa and fibroblast cells were thawed and cultured for 2-6 days after confluency, without serum starvation, before whole cell injection. They were, thus, presumably at G₀ phase of the cell cycle. Immediately before whole cell injection, donor cells were trypsinized, washed by centrifugation, and re-suspended in injection medium of TL-HEPES and 10% polyvinylpyrrolidone (PVP) solution at 1:1.

### Example 2

### Enucleation and Whole Cell Injection.

Recipient oocytes were prepared by centrifugation for 10 minutes in an Eppendorf Centrifuge at 12,000 g in 200 μL TL-HEPES medium to allow detection of the first polar body. Only oocytes with excellent morphology and a visible polar body were selected for this experiment. For enucleation, groups of oocytes were transferred into a droplet of TL-HEPES containing 5 μg/mL cytochalasin B (CB), which had previously been placed in the operation chamber on the microscope stage. In the initial experiments, enucleation was accomplished by aspiration of the first polar body and the metaphase II plate in a small amount (<15% of the oocyte volume) of cytoplasm. Successful enucleation was confirmed by examination after staining with 5 μg/mL Hoechst 33342. The enucleation protocol was later improved by partial zona dissection near the polar body and then pressing out cytoplasm at the dissection area [Kubota, C., et al. “Six cloned calves produced form adult fibroblast cells after long-term culture,” Proc. Natl. Acad. Sci. USA 97, 990-995 (2000).] Successful enucleation was confirmed by staining the isolated cytoplasm.

Whole cell injection was accomplished by using an injection pipette with a sharp, beveled tip (inner diameter 10-25 μm depending on cell type). Single cells were transferred to injection medium and kept at room temperature for up to 3 hours before injection (FIG. 4A, B and C). In FIG. 4(A), a fibroblast cell (arrowhead) is aspirated into the injection pipette. In FIG. 4(B), the cell is expelled into the cytoplasm of the enucleated oocyte. Shown in (C) is the verification of the absence of the donor cell in the injection pipette. (D) shows hatched blastocysts (Day 6) produced by whole cell injection of fibroblast cells.

### Example 3

### Activation of Oocytes.

Electrical stimulation was as described in the literature [Lee, J-W., Kim, N-H., Lee, H-T., & Chung, K-S., “Microtubule and chromatin organization during the first cell-cycle following intracytoplasmic injection of round spermatid into porcine oocytes.” Mol. Reprod. Dev. 50, 221-228 (1998).], with slight modifications. Briefly, reconstructed embryos were washed and pre-incubated for 20 seconds in activation medium (0.25 M mannitol solution supplemented with 0.01% polyvinyl alcohol, 0.5 mM HEPES, 0.1 mM CaCl₂.H₂O and 0.1 mM MgCl₂.6H₂O with a pH of 7.2) at room temperature. Electrical stimulation was delivered with a BTX Electro Cell Manipulator (Biotechnologies and Experimental Research, Inc., San Diego, Calif.) to a chamber with two parallel platinum wire electrodes (200 μm outer diameter) spaced 1 mm apart overlaid with activation medium. The reconstructed oocytes were exposed to an electrical pulse for 10 seconds at 5V AC followed by a 1×30 μsec pulse at 2.2 kV/cm DC at room temperature. Non-manipulated, but UV exposed, oocytes were activated 3 hours after UV exposure as a control. Following somatic-cell injections, oocytes were either immediately activated and then cultured in NCSU23 medium containing 10 μg/mL CB and cycloheximide (CH) for 5 hours, or left in NCSU23 medium at 38.5° C. under 5% CO₂ in air for 1.5-, 3- and 6-hour periods before electrical activation treatment.

Investigations were conducted to determine the conditioning requirements of the injected whole cells involving testing whether an extended time interval between whole cell injection and oocyte activation would benefit reprogramming and development of reconstructed embryos. Of particular interest, was whether different donor cell types (cumulus, mural granulosa, and fibroblast cells) require different exposure times (0, 1.5, 3 and 6 hours) in the metaphase II-stage (MII) oocyte cytoplasm (see FIG. 2). No blastocyst development was observed when activation was conducted immediately after whole cell injection (0 hours), regardless of donor cell types. Activation at 6 hours post-injection also produced low blastocyst development for all cell types tested. Optimal development was observed when activation was applied at 1.5 hours post cell injection for cumulus and mural granulosa cells (18% and 16%, respectively), and at 3.0 hours for fibroblast cells (19%). In mice, delayed activation from 1-6 hours after injection of the isolated nucleus was also found beneficial to embryo development (Wakayama, T., et al., 1998). Fibroblast cells were observed to require longer exposure to the MII cytoplasm than granulosa cells when whole cells were used for injection. These results point to the possibility that the plasma membranes of different donor cell types require different amounts of time for dissolution. This phenomenon is consistent with the observation that different fusion rates are associated with fibroblast and cumulus cells in the cell fusion method of cloning. Both of these phenomena may also be attributable to the differences in the membrane properties between these two cell types.

### Example 4

### Effect of UV Irradiation on Cloned Oocytes.

In order to further improve the development rate of cloned embryos by whole cell injection, investigations were conducted to determine whether removing UV-light exposure from the cloning protocol would improve its efficiency. It was observed that UV exposure during oocyte enucleation had a significant and detrimental effect on embryo development. When Hoechst stained oocytes were exposed to UV during enucleation, blastocyst development by cloned embryos was significantly lower (19%; Table 1) than those produced without UV exposure (37%; P<0.05). The UV exposure also detrimentally affected the development of control parthenogenetically activated oocytes. The blastocyst rate of parthenotes without UV exposure (48%) was significantly higher than those with UV exposure (25%). The present results warn against the use of Hoechst staining and UV exposure during enucleation which has been the common practice for cloning (Betthauser, J., 2000, including title of the reference; Westhusin, M. E., et al., “Viable embryos and normal calves after nuclear transfer into Hoechst-stained enucleated demi-oocytes of cows,” J. Reprod. Fert. 95: 475-480, 1992). This is also consistent with previous observations that UV exposure of oocytes could cause abnormal meiosis and poor development. Bradshaw, J., et al., “UV irradiation of chromosomal DNA and its effect upon MPF and meiosis in mammalian oocytes,” Mol. Reprod. Dev. 41: 503-512 (1995); Dominko, T. et al., “Bovine oocyte cytoplasm supports development of embryos produced by nuclear transfer of somatic cell nuclei from various mammalian species,” Biol. Reprod. 60: 1496-1502 (1999); Tao, T., et al., “Optimisation of porcine oocyte activation following nuclear transfer,” Zygote 8: 69-77 (2000).

Values provided in Table 1 below with different superscripts within each column differ at a statistically significant level (P<0.05). Oocytes in the NT+UV (no treatment, with UV exposure) group were stained for DNA and enucleated under UV light. The UV exposure time was <10 seconds. Oocytes in the activation+UV group were stained for DNA and exposed to UV light for the same amount of time as those in the NT group.

TABLE 1
Effects of UV exposure during enucleation on development of
cloned and activated oocytes after 7 days of in vitro culture

| | Number | No. (%) | | | | Cell |
|---|---|---|---|---|---|---|
| | oocytes | survived | Number | No. (%) | No. (%) | number |
| Treatment | injected | injection | activated | cleaved | blastocysts | (mean ± SEM) |
| NT + UV | 125 | 115 | 105 | 48 (45.7) | 20 (19.0)^{a} | 28 ± 4 |
| | | (92.0) | | | | |
| NT − UV | 125 | 112 | 110 | 71 (64.5) | 41 (37.3)^{b,c} | 37 ± 5 |
| | | (89.6) | | | | |
| Activation + | — | | 125 | 95 (76.0) | 31 (24.8)^{a,b} | 30 ± 4 |
| UV | | | | | | |
| Activation − | — | | 125 | 107 (85.6) | 60 (48.0)^{c} | 40 ± 5 |
| UV | | | | | | |

Statistical analyses. Differences in the percentages of oocytes developing to a particular stage were determined by Chi-square analysis.

### Example 5

### In Vitro Culture of Reconstructed Embryos and Parthenotes.

After activation treatments, the reconstructed, and control embryos were thoroughly washed and cultured in 50 μL drops of NCSU23 supplemented with 1% MEM non-essential amino acid and 0.4 mg/mL bovine serum albumin (BSA) for 7 days at 38.5° C. in 5% CO₂ in air without a medium change. The rates of activation, cleavage and development to blastocyst were examined on day 2 and 7 after activation, respectively.

Assessment of Whole Cell Injection and Embryonic Development.

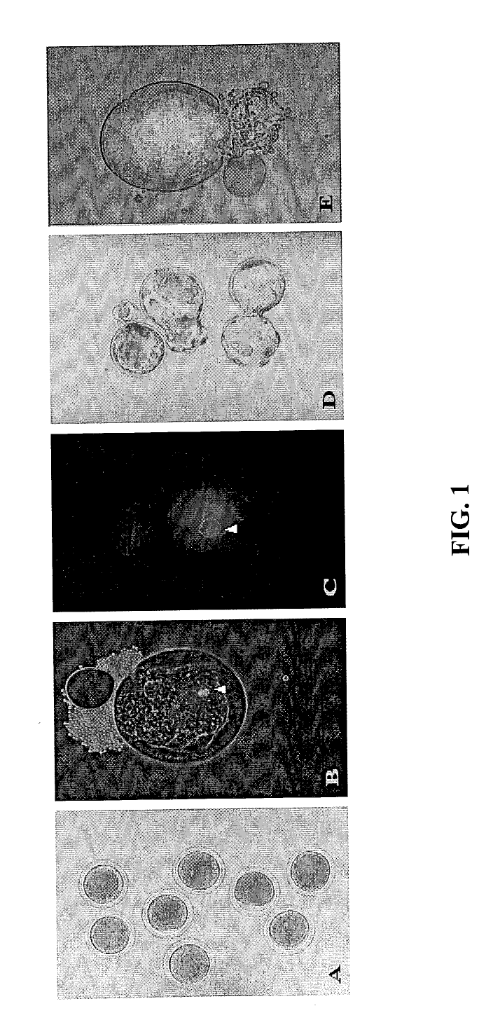

To assess the extent of success in injection of whole donor cells into the cytoplasm of enucleated oocytes, the present inventors investigated whether, and when, the enucleated oocytes break down the plasma membrane and form pronuclei from an injected whole cell. Fibroblast cells whose plasma membranes were stained with a live-membrane fluorescent dye prior to injection were injected into enucleated oocytes. The cell membranes were stained with PKH67 Green Fluorescent cell liner kit (PKH67-GL). Immediately after whole cell injection, the membrane of the injected fibroblast cell was seen to be intact and emitted green fluorescence (see FIG. 1A). The injected cells were visible in the oocytes' cytoplasm within 3 h following injection. To assess chromatin remodeling, oocytes were stained with Hoechst 33342 dye 6, 12, and 24 hours after activation. Swollen nuclei or distinct pseudo-pronuclei in enucleated cytoplasm were considered as having been activated. Oocytes, 7 days after activation, were fixed and stained with 5 μg/mL of Hoechst 33342 to assess embryonic development. The cell number for each fixed embryo was counted and its developmental stage recorded.

Six hours after oocyte activation, the plasma membrane of the injected fibroblast cell became undetectable, while the nucleus, stained with Hoechst 33342, was clearly visible (see FIG. 1B). Full nucleus swelling (arrowhead) was observed 12 h after oocyte activation. (see FIG. 1C). The injected whole cell was competent to support development to the hatched blastocyst stage in vitro, as demonstrated in FIGS. 1D and E. These observations demonstrated that the recipient oocytes were capable of dissolving the plasma membrane and reprogramming the nuclei of the injected donor cells.

### Example 6

### Superovulation and Embryo Transfer.

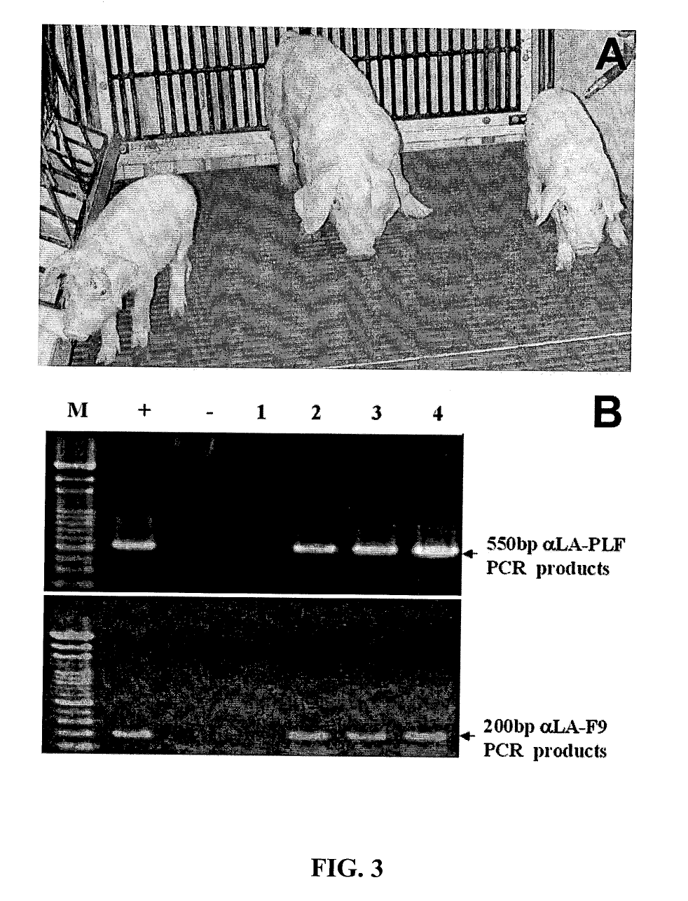

After establishing the optimum conditions for cloning by whole cell injection, in vivo matured oocytes were used for the production of cloned piglets. Fibroblasts used for whole cell injection were derived from the ear of a sow carrying two transgenes, pLF and hFIX, both driven by the lactoalbumin promoter (αLA). A total of 685 whole-cell injected oocytes were transferred to 9 recipient pigs on Day 1 of the estrous cycle (see Table 2). Six of the recipients (67%) were confirmed pregnant by ultrasound 21 days after embryo transfer. Six piglets were aborted from three recipients at days 23 to 28 of gestation, and four live piglets were born from the remaining three recipients by C-section on Feb 15, March 23 and Apr. 7, 2002 (see Table 2). However, one piglet died three days after birth due to infection and abnormal spine development. All three live piglets were examined by veterinarians and were found to be active, and healthy, with no apparent birth defects. All live born (see FIG. 3A) and aborted piglets were tested positive for both transgenes by polymerase chain reaction (as shown in FIG. 3B, showing PCR assay for the αLA-pLF and αLA-hFIX double transgenes), which results confirmed that they were derived from the donor sow. Expected fragments of 550 bp for pLF and 200 bp for hFIX were obtained; Lane 1: Recipient pig; Lane 2: Placenta of a cloned fetus; Lane 3: umbilical cord of a cloned fetus; Lane 4: Donor cells; (+): positive control; (−): negative control; and (M): 100 bp markers. See Example 7, below.

Pubertal crossbred gilts, aged 8 to 10 months, were synchronized with Regumate (containing 0.4% altrenogest; 20 mg/day; Intervet, Boxmeer, Netherlands) mixed in commercial feed and given each morning for 15 days. All donor gilts were injected with 2,000 IU PMSG (Folligon & Chorulon) and 80 hours later with 1,500 IU hCG (Folligon & Chorulon). Recipient gilts were injected with half the dosage of PMSG and hCG administered to the donors. Oocytes were surgically collected 44-46 hours after hCG injection by flushing from the oviduct with Dulbecco's Phosphate Buffer Saline. (Gibco BRL, Cat. No.11500-030). To produce cloned pigs, reconstructed embryos were surgically transferred into the oviducts of synchronized foster mothers 20-24 hours after activation. An ultrasound scanner (Aloka SSD-500, JAPAN) with an attached 3.5 MHz transabdominal probe was used to check pregnancies 20-21 days after embryo transfer. Pregnant recipients were reexamined by ultrasound around the time of the first to second estrous cycle and again 30 days before the expected due date.

Statistics for overall pregnancy rates of cloned embryos are provided in Table 2, below.

TABLE 2
Embryo Transfer and pregnancy rates of whole
cell injected embryos cloned from skin fibroblasts
from a double transgenic sow (αLA-pLF and αLA-hFIX)

| No. oocytes collected | 1036 | |
|---|---|---|
| No. (%) oocytes enucleated | 893 | (83) |
| No. (%) oocytes injected | 801 | (77) |
| No. (%) embryos cultured | 718 | (69) |
| No. (%) embryos transferred | 685 | (66) |
| No. (%) recipients | 6/9 | (67) |
| pregnant/total | | |
| No. recipients furrowed | 3 | |
| No. (%) cloned piglets born | 4 | (0.4) |

### Example 7

### Detection of Transgene.

The umbilical cords and placental tissues were homogenized in 700 μL lysis buffer (50 mM Tris-HCl, 100 mM EDTA, 100 mM NaCl, pH 8.0) containing 500 μg of proteinase K and 70 μL of 10% SDS, and were then incubated at 58° C. for 16-20 hours. Primers, corresponding to αLA-pLF (5′ CCT AGA ACC AAC ACT ACC AG; 3′ AGA AGC CCT CCT TAT GCA GA (SEQ ID NO: 1)) and αLA-hFIX (5′ GTG ACC CCA TTT CAG AAT CTT G (SEQ ID NO: 2); 3′ CCG ATT CAG AAT TTT GTT GGC) (SEQ ID NO: 3), were employed to amplify 550 base pairs (BP) and 200 bp of respective fragments from the junction region of the transgenes. PCR reactions were performed for 30 cycles with denaturation at 94° C. for 30 seconds, annealing at 55° C. for 1 minute and extension at 72° C. for 1 minute in a thermal cycler (AG-9600: AcuGen Systems, USA). The reaction mixture was then analyzed on a 2% agarose gel, followed by staining with ethidium bromide. The amplified DNA bands were then visualized by ultraviolet transillumination.

What is claimed is:

1. A novel method for the production of a reconstructed oocyte, the method comprising the steps of:
(a) selecting one or more recipient oocytes from a mammal of a specific species;
(b) enucleating the selected recipient oocytes;
(c) selecting one or more somatic donor cells from a donor cell source;
(d) injecting a whole cell from the one or more donor cells into an enucleated oocyte to form a reconstructed oocyte; and
(e) culturing the reconstructed oocyte under conditions sufficient to insure development of the reconstructed oocyte to a further developmental stage.

2. The method of claim 1, wherein the donor cells are selected from the group consisting of cumulus cells, mural granulosa cells, and fibroblast cells.

3. The method of claim 1, wherein the donor cell source is a stable cell line.

4. The method of claim 1, wherein the donor cell source is a mammal that has reached a developmental stage of independent viability.

5. The method of claim 4, wherein the mammal is a transgenic mammal.

6. The method of claim 1, wherein the donor cell source is selected from the group consisting of an embryo and fetal tissue.

7. The method of claim 1, wherein the species of mammal is selected from the group consisting of pig, rabbit, cattle, goat and mouse.

8. The method of claim 1, wherein the method includes the further step of centrifugation of the donor oocytes prior to enucleation.

9. The method of claim 1, wherein the method includes the further step of activating the reconstructed oocyte at a time subsequent to formation of the reconstructed oocyte sufficient to result in optimization of cloning efficiency.

10. The method of claim 9, wherein the reconstructed oocyte is activated by electrical stimulation.

11. The method of claim 9, wherein the reconstructed oocyte is activated while minimizing exposure of the reconstructed oocyte to ultraviolet (UV) radiation.

12. The method of claim 9, wherein the step of activating the reconstructed oocyte occurs from 0 to 10 hours after injection of the donor cell into the enucleated oocyte.

13. The method of claim 12, wherein activation occurs from 1 to 6 hours after injection of the donor cell into the enucleated oocyte.

14. The method of claim 1, wherein the method includes the additional step of conditioning the donor cells prior to activation.

15. A cloned mammal produced from a reconstructed oocyte obtained by the method of claim 1.

16. A stable cell line derived from a reconstructed oocyte obtained by the method of claim 1.

17. An embryo developed from a reconstructed oocyte obtained by the method of claim 1.

18. Stem cells developed from a reconstructed oocyte obtained by the method of claim 1.

19. Tissue developed from a reconstructed oocyte obtained by the method of claim 1.

20. An organ developed from a reconstructed oocyte obtained by the method of claim 1.

21. The method of claim 1, wherein the method further comprises the step of altering one or more nucleotide sequences of the donor cell by genetic engineering techniques.

22. A cloned mammal developed from a reconstructed oocyte obtained by the method of claim 21.

23. The cloned mammal of claim 22, wherein the mammal displays a desirable phenotypic trait conferred on the mammal through the altered nucleotide sequence.

24. The mammal of claim 23, wherein the one or more desirable phenotypic traits comprise a reduced immunostimulatory effect on a pre-selected potential xenotransplantation organ, tissue or cell recipient.

25. The method of claim 23, wherein the desirable phenotypic trait comprises production of one or more pharmaceutically active species.

26. A method for the production of donor material comprising cells, tissue or organs for xenotransplantation, the method comprising the steps of:
(a) producing a cloned donor source according to the method of claim 1; and
(b) harvesting the cells, tissue or one or more organs from the cloned donor source.

27. The method of claim 26, wherein the method comprises the further step of altering at least one nucleotide sequence of one or more cells derived from the donor material by genetic engineering techniques.

28. A method for the production of donor cells, tissues, or organs for xenotransplantation, the method comprising the steps of:
(a) producing a cloned donor mammal according to the method of claim 21; and
(b) harvesting a cell, tissue, or organ from the cloned mammal for xenotransplantation.

29. A method for the production of one or more potentially therapeutic proteins comprising the steps of (a) producing a cloned mammal according to the method of claim 21, wherein the desirable phenotypic trait comprises expression of the one or more proteins, and (b) extracting the one or more proteins from the cloned mammal.

30. The method of claim 1, wherein the developmental stage to which the reconstructed oocyte is developed is an embryo stage, and wherein the method comprises the further step of transplanting the embryo into a surrogate mother.

31. The method of claim 29, wherein the steps further comprise
(a) maintaining the surrogate mother in which the embryo was implanted under conditions sufficient to insure development of the embryo into a fetus capable of sustaining life outside of the surrogate mother; and
(b) delivering the developed fetus to produce a cloned mammal.
